(19) United States
(12) Patent Application Publication
(10) Pub. No.: US 2013/0023896 A1
(43) Pub. Date: Jan. 24, 2013

(54) SURGICAL MANIPULATION AND OCCLUSION DEVICE

(71) Applicant: Jennifer C. Quimby, Poulsbo, WA (US)

(72) Inventor: Jennifer C. Quimby, Poulsbo, WA (US)

(21) Appl. No.: 13/554,960

(22) Filed: Jul. 20, 2012

Related U.S. Application Data
(60) Provisional application No. 61/510,008, filed on Jul. 20, 2011.

(51) Int. Cl. A61B 17/42

(52) U.S. Cl. 606/119

(57) ABSTRACT

Provided are uterine manipulators comprising in combination: a vaginal occlusion portion comprising a resilient deformable member (e.g., bulb, cone, cylinder, etc.) having a channel therethrough having a proximal opening sized to fit, during use of the device, snuggly against a manipulation rod or tube, and a relatively larger distal opening suitable to provide for angular and/or rotational movement of the rod or tube passing through the member via the distal and proximal openings while minimizing movement of the vaginal occlusion member when the tube is subjected to manipulation, and wherein the member is suitably configured to fit snugly against the contours of an inner vaginal tissue surface; and a cervical fitting portion which is securable to the cervix. Also provided are surgical methods using said elements to provide for a snug fit with the cervix while sealing the vagina, wherein maintaining abdominal insufflation during surgery is afforded.

### CROSS-REFERENCE TO RELATED APPLICATIONS

This application claims the benefit of priority to U.S. Provisional Patent Application Ser. No. 61/510,008 filed 20 Jul. 2011, which is incorporated by reference herein in its entirety.

### FIELD OF THE INVENTION

Aspects of the invention relate generally to medical devices, and more particularly to surgical instrumentation for moving and positioning a uterus for better visualization and access during surgery. Additional aspects relate to such surgical instrumentation that additionally occludes the vagina to prevent loss of insufflation during surgery. Further aspects relate to surgical methods as described herein.

### BACKGROUND

The most feared complications when performing a hysterectomy (removal of uterus) by any route of approach are potential urinary tract lesions including bladder, and particularly the ureters. The incidence of urinary tract injury in laparoscopic hysterectomy is about 0.02-1.7%, primarily arising during securing of the uterine vessels, because the zone for tying the uterine artery is located near the junction with the ureter and the bladder.

Total laparoscopic hysterectomy (HTL) is a significant challenge for laparoscopic surgeons because the surgery is technically difficult, demanding, and often is accompanied by prolonged surgical time, thereby making the incidence of complications is greater.

HTL is facilitated by use of devices such as a uterine manipulator to remove the complete human uterus, reducing the time and the incidence of surgical complications. There are currently several tools to remove the uterus by laparoscopy, and related surgical procedures involving expanding the patient's abdomen through the introduction of an innocuous gas (insufflation, e.g., usually with carbon dioxide) to facilitate the surgery.

Use of some type of uterine manipulator is currently deemed essential by practitioners for all laparoscopies involving the female pelvic organs (uterus, tubes, ovaries) when a uterus is present. Surgery without a uterine manipulator is more dangerous and can be more time consuming. Exemplary laparoscopies in which a uterine manipulator has substantial utility include: tubal ligations; diagnostic laparoscopies for evaluating pelvic pain and infertility; treatment of endometriosis, removal of pelvic scars (adhesions) involving the uterus, fallopian tubes and ovaries; treatment of ectopic pregnancy; removal of uterine fibroids; removal of ovarian cysts; removal of ovaries; tubal repair; laparoscopic hysterectomy, laparoscopic repair of pelvic bowel or bladder; sampling of pelvic lymph nodes; “tying up” the bladder to prevent urine loss; and biopsy of pelvic masses.

Most manipulators currently sold and used are merely rigid instruments which attach in a fixed manner to the uterus, and protrude from the vagina. The instrument is typically held in place by sharp hooks embedded in the cervix, such as with a tenaculum. A typical example of such devices is the Majoli Uterine Manipulator/Injector, sold by Cook Urological, Inc. of Spencer, Ind. (U.S. Pat. No. 5,643,311), The handle of the instrument is grasped outside the vagina, and uterine manipulation is affected by applying torque to the rigid handle of the device at a point some distance from the organ. The uterus typically may be elevated only 45° from the vaginal axis, or lowered 10°-15°. The capability for movement to the right or left is minimal, and pelvic tissues and organs are unnecessarily stressed by application of excessive torque. Exposure of the vital regions of the pelvis is difficult, and surgery with such instruments is often suboptimal or even unsafe. Further, such devices and methods routinely require an extra staff member to maintain the instrument in the correct position to perform a procedure. In some cases this is very cumbersome, and obviously adds time and cost to the procedure.

Another potential complication is uterine perforation resulting from excessive force being exerted by the physician when trying to place the manipulator into the uterus or during attempted manipulation of the uterus for better visualization.

In an attempt to avoid the above problems, a more recent class of manipulators has been developed. Examples of this class are the Harris Uterine Manipulator Injector (HUMI) disclosed in U.S. Pat. No. 4,430,076, and similar devices marketed as the Zinnanti Uterine Manipulator Injector (ZUMI) and the Kronner manipulator. The HUMI was the first plastic, disposable manipulator. The HUMI uses an intrauterine balloon and external spring and stop at the cervical os.

Another device of this type, which uses two balloons mounted on a long, stiff metal rod, one inside and one outside of the uterus, is marketed by C. R. Bard, Inc. of Tewksbury, Mass.

Another uterine manipulator is the McCartney tube (U.S. Pat. No. 8,082,925; FIG. 2) consisting of a cylindrical, slightly curved tube, which at one end has a lid with a valve whose function is to prevent gas from leaking, and at the opposite end has a rounded smooth edge, whose role is to expose the endopelvic fascia. However, this device has the disadvantage that it is simply a silastic tube in which one of its edges serves to demarcate the edge of the fascia to carry out the colpotomy (incision) and to prevent leakage of pneumoperitoneum, but said device does not secure the cervix and uterus and allow for manipulation of the uterus, so that it does not facilitate the procedure because it fails to provide an optimal view of the vulnerable structures, which disadvantage is more evident in large uteri. In addition, the valve whose function is to prevent gas from leaking from the abdomen, in practice does not prevent this leakage, and moreover the annular external ribs (shown as element 132 in FIG. 2 herein) are also not adequate for this purpose.

Another known device being currently used for the same purpose is the Wattiez manipulator (Clermont-Ferrand) (U.S. Pat. No. D421,497; FIG. 1) consisting of a long cylindrical tube, which carries at one end an inverted L-shaped, rotatable tongue having rounded smooth edges, adapted to expose the endopelvic fascia at the opposite end of the tube, proximal to the terminal portion is a short, rectangular sleeve-shaped device, whose function is to provide rotary movements to the tube and thus to the tab located on, the other end. Inside said tube, a cylindrical rod is housed, which at the end where the tube tongue is located has a threaded portion to which externally threaded conical tips are adapted to be fixed to the cervical canal; a rectangular handle is located at the opposite end of the cylindrical rod, which is adapted to move the inner rod. In the middle of the outer tube, a circular and conical sliding device comprising three soft rings is disposed to facilitate occlusion of the vagina and allegedly prevent escape of gas. However, this uterine manipulator has the disadvantage that it is very large and difficult to maneuver, and for the exposure of the fascia it has a rotary latch instead of a complete cap, so the exposure and protection of structures such as the bladder and uterine vessels may be insecure and not uniform. In addition, the three soft rings do not adequately prevent gas escaping from the abdomen because they are rigidly attached to the device and therefore they do not allow the physician to manipulate the device as required during surgery without the rings moving from the places of contact within the vagina and thus allow gas to escape.

Another instrument of this type is known as RUMI system that is a uterine injector manipulator consisting of two main components: 1.—a reusable handle and 2.—a single use, disposable sterile tip that adheres to said handle. The handle consists of a grip element, a rod, a clamping drum and a blocking trigger. The handle has a “L” shaped design and 20 cm. (8″) long along the rod and 10 cm. (4″) long along the grip element. The drum (placed at the extreme position of said rod) is used to position the tip and rotate it into an arc of 140 degrees. The grip element of manipulator is 90 degrees relative to the rod. By turning the grip element, while the blocking trigger is pressed, allows the tip positioning drum to rotate and thus to change the position of the tip. When released, the blocking trigger secures the position of rotation of the grip element and the clamping drum. The single use tip and associated body is manufactured in silicone USP Class IV medical grade, an internal wire for support and a balloon fastened from outside at the distal end of the tip, which is also manufactured in silicone USP Class IV. When properly inflated, the balloon positions the tip in the uterus. The tips have three delivery ports along the arrow thereof. One port is for inflating the balloon with sterile saline. The remaining two ports near the distal end position of the tip are for administrating contrast media. Two silicone catheters extended from the tip serve as balloon extensions and for delivering the dye into the cavity. The balloon catheter, 30 cm. (12″) long, has a white syringe connection, a hose clamp and a white luer connector. The clamp is operated manually to open or close the closure seal of the globe. The catheter of the contrast medium is clear in color and 60 cm. (24″) long and it has a white luer connector to adjust the syringe of the dye. To accommodate the different uterine sizes, the tips are provided in different diameters and lengths of the arrows.

Another variant of this device is known as pneumo-occluder system consisting of the RUMI system (see U.S. Pat. No. 5,643,285 and U.S. Pat. No. 5,520,689) adapted to include a cap in the proximal end, which press fits to delimit the endopelvic fascia and an inflatable silicone ring being placed (apart from the vaginal extender with cevix-engaging base) at the middle portion of the instrument inside the vagina so as to occlude this latter and prevent leakage of gas. It is an instrument primarily designed for mobilizing the uterus, and has very fragile elements so its use to remove large uterus is very difficult. In addition, the inflatable silicone ring (43 shown in FIG. 3 herein) is rigid and attached at its base across the entire width of the ring base such that angular movement of the device, being immediately transferred to the ring, tend to break the seal allowing loss of insufflation, and thus does not adequate occlude the vagina.

The Hohl's uterine manipulator (US Patent Application 20050085827) is another of the tools currently used in surgical procedures. This manipulator comprises a non-hollow, long cylindrical device adapted to include at one end a conical-shaped threaded cone to fix in the cervical canal, said device has in turn an extensor coupled to the tip thereof. This cylindrical device slides within a T-shaped hollow cylindrical tube, which includes a cap fastened to the end opposite to the “T” by means of an external threaded portion and nut connection. At the T-shaped end, a screw is provided inside the “T” to fix the internal cylindrical rod. This manipulator has the disadvantage that it is completely straight and lacks of articulation in the cap area so that no anteflexion and no 90 degree movements are available, hence in some difficult patients and in certain technical difficulty circumstances, the access to the rear of the fascia can be very difficult or even dangerous. In addition, there is no system or element in place to occlude the vagina properly, and instead gas is allowed to escape.

One of the most significant complications of the aforementioned type of manipulators is that upon a few surgical cuts around the uterus, so that the uterus can be removed, the gas that was used to insufflate the abdomen rushes out of the vagina. This results in reducing the ability of the surgeon to complete the hysterectomy.

Although progress has been made in the art as noted above, all existing uterine manipulators known to the inventor possess significant disadvantages and limitations, including maintaining sufficient insufflation of the abdomen during laparoscopic hysterectomies. With the increasing pressure for cost containment in medical treatment and the increasing popularity of laparoscopic gynecological procedures moving to outpatient clinics and gynecological offices, a cost-effective uterine manipulator having enhanced manipulation capability, proper maintenance of insufflation, and ease of use would be very attractive to the medical profession.

Therefore, there is a need in the art for devices that provide for better occlusion of vaginal opening to maintain insufflation of the abdomen and yet provide for adequate uterine manipulation during surgery.

### SUMMARY OF ASPECTS OF THE INVENTION

The following embodiments and aspects thereof are described and illustrated in conjunction with compositions and methods which are meant to be exemplary and illustrative, not limiting in scope.

Particular aspects provide medical instrumentation, and in specific aspects, a highly maneuverable device configured to allow adequate exposure of all anatomic structures within the region, for moving the uterus to different positions for optimal visualization and surgical access. The device simultaneously occludes the vagina to block gases from escaping through it (prevents loss of pneumoperitoneum).

Aspects of the present invention provide a cost-effective uterine manipulator having enhanced manipulation capability, and that provides for maintenance of insufflation of the abdomen by substantially improved occlusion of vaginal opening during surgery, and ease of use would be very attractive to the medical profession.

Particular aspects provide a uterine manipulator device to be used in laparoscopic surgical procedures, comprising: a uterine occlusion element, or elements, comprising a distal cervical fitting portion to secure the uterus and cervix and expose the fascia, and a proximal vaginal occlusion portion (optionally separated from the cervical fitting/securing portion) configured to seal the vagina. Particular embodiments comprise an inflatable balloon at a distal trans-cervical end of a tubing portion that extends through the length of the device including through the uterine occlusion element(s), the tubing providing for fluid communication between a fluid inlet at a proximal exterior end and the balloon to enable intrauterine inflation of the balloon with a fluid, wherein the uterine occlusion element(s) is/are positioned or positionable along the tubing portion between the cervix and the vagina to provide for a snug fit with, and securing of the cervix while also providing for sealing the vagina by the proximal vaginal occlusion portion, thereby preventing leakage of pneumoperitoneum (loss of insufflation) during a surgical procedure (e.g., laproscropic procedure).

In certain aspects, the cervical fitting/securing portion is configured to fit snugly against the cervix, and further comprises a barb-containing portion configured to engage and secure the cervix to preclude or retard inadvertent detachment slipping or rotating of the occlusion device relative to the cervix.

In particular aspects, the surgery comprises laparoscopic surgery. In certain aspects, the surgery comprises hysterectomy.

Additional aspects provide a method of conducting surgery, comprising: obtaining a uterine manipulator comprising a uterine occlusion element, or elements, comprising a distal cervical fitting portion to secure the uterus and cervix and a proximal vaginal occlusion portion (optionally separated from the cervical fitting/securing portion) configured to simultaneously expose the fascia and seal the vagina; and positioning, within a patient's vagina, the uterine occlusion element, or elements between the cervix and the vaginal opening to provide for a snug fit with, and securing of the cervix while also providing for sealing the vagina by the proximal vaginal occlusion portion, wherein maintaining insufflation of the patient's abdomen during a surgical procedure is afforded. In certain aspects, the distal cervical fitting portion of the uterine occlusion element, or elements, is/are capable of being affixed to the cervix via securing barbs to secure the cervical tissue to preclude or retard inadvertent detachment, slipping or rotating of the uterine occlusion element, or elements, relative to the cervix and uterus during surgery. In these regards, the unique configuration of the vaginal occlusion element, which, as described in more detail hereunder, provides for angular movement/adjustment of the device tubing and uterus, without concomitant dislodgment of the vaginal seal aspect, provides a substantial improvement with respect to preventing leakage of pneumoperitoneum (loss of insufflation) during surgery, and the combination of this seal-preserving configuration plus the unique cervical fitting/securing portion having securing barbs (or the like as described herein) provide for an optimal surgical instrument (uterine manipulator) that solves a long-standing problem in the art; namely, how to adequately prevent leakage of pneumoperitoneum (loss of insufflation) while maintaining optimal control of cervical/uterine manipulation during surgery.

In particular aspects, the surgery comprises laparoscopic surgery. In certain aspects, the surgery comprises hysterectomy.

Certain aspects provide a vaginal occlusion device, comprising in combination: a vaginal occlusion portion comprising a resilient deformable member (e.g., bulb, cone, cylinder, etc.) having a channel therethrough having a proximal opening sized to fit, during use of the device, snuggly against a manipulation rod or tube, and a relatively larger distal opening suitable to provide for angular and/or rotational movement of the rod or tube passing through the member via the distal and proximal openings while minimizing movement of the vaginal occlusion member when the tube is subjected to manipulation, and wherein the member is suitably configured to fit snugly against the contours of an inner vaginal tissue surface, and a cervical fitting member configured to, in use of the device, engage and secure a cervix. In particular embodiments, in use of the device, the vaginal occlusion portion is axially slidably positionable away from the cervical fitting member along the rod or tube. In certain aspects, at least a part of the tubing portion is rigid. In particular embodiments, the cervical fitting member is configured to fit snugly against the cervix and secure the cervix. In preferred embodiments, securing the cervical fitting member occurs via barb means, wherein the barb means are configured to preclude or retard slipping or rotating of the cervical fitting member relative to the cervix. In certain aspects, the resilient deformable member generally comprises at least one shape selected from the group consisting of a sphere, a cylinder, a cone, a torus, a bulb, and a wheel.

Additional aspects provide a uterine manipulator suitable for laparoscopic surgical procedures, comprising: a vaginal occlusion portion comprising a resilient deformable member (e.g., bulb, cone, cylinder, etc.) having a channel therethrough having a proximal opening sized to fit, during use of the device, snuggly against a manipulation rod or tube, and a relatively larger distal opening suitable to provide for angular and/or rotational movement of the rod or tube passing through the member via the distal and proximal openings while minimizing movement of the vaginal occlusion member when the tube is subjected to manipulation, and wherein the member is suitably configured to fit snugly against the contours of an inner vaginal tissue surface; a cervical fitting member configured to be securable, in use, to a cervix; a balloon at a distal trans-cervical end of a tubing portion that extends through the vaginal occlusion element and the cervical fitting element, the tubing in fluid communication between a fluid inlet at a proximal exterior end and the balloon to enable intrauterine inflation of the balloon with a fluid, wherein the vaginal occlusion element is positioned or positionable along the tubing portion between the cervix and the vagina to provide for a snug fit with the vagina, thereby preventing leakage of pneumoperitoneum during a surgical procedure. In certain embodiments, securing the cervical fitting member occurs via barb means, wherein the barb means are configured to preclude or retard slipping or rotating of the vaginal occlusion device relative to the cervix. In particular aspects, the deployment of the barbs allows for a secure fitting tight up against and/or within the cervix. In certain embodiments, a proximal exterior end of the device comprises a handle, and in certain aspects, the handle is in mechanical communication with the cervical fitting member. In particular embodiments, the handle is configured to deploy or otherwise manipulate or position the barb means of the cervical fitting member, wherein the barb means prevent or retard slipping or rotating of the device from placement, and wherein, the handle is in mechanical communication with the cervical fitting member. In certain aspects, the deployment of the barb means is controlled by a switch or a spring. In certain embodiments, the resilient deformable member comprises the shape of at least one shape selected from the group consisting of a cylinder, a cone, a torus, a bulb, and a wheel. In certain aspects, the surgery comprises laparoscopic surgery (e.g., hysterectomy).

Further provided is a vaginal occlusion device, comprising: a vaginal occlusion portion comprising a resilient deformable member (e.g., bulb, cone, cylinder, etc.) having a channel therethrough having a proximal opening sized to fit, during use of the device, snuggly against a manipulation rod or tube, and a relatively larger distal opening suitable to provide for angular and/or rotational movement of the rod or tube passing through the member via the distal and proximal openings while minimizing movement of the vaginal occlusion member when the tube is subjected to manipulation, and wherein the member is suitably configured to fit snugly against the contours of an inner vaginal tissue surface. In certain aspects the vaginal occlusion device is in combination with a cervical fitting member suitable to secure a cervix and a uterine manipulation rod or tube. In particular aspects, the vaginal occlusion portion is positionable along the rod or tube. In certain embodiments, at least a part of the rod or tubing portion is rigid. In certain embodiments, the cervical fitting member is configured to fit snugly against the cervix and secure the cervix using barb means. In particular aspects, securing the cervical fitting member occurs via barbs, wherein the barbs are configured to preclude or retard slipping or rotating of the vaginal occlusion device relative to the cervix. In certain embodiments, the resilient deformable member comprises a shape selected from at least one from the group consisting of a cylinder, a cone, a torus, a bulb, and a wheel.

Additionally provided are methods for conducting surgery, comprising: obtaining a vaginal occlusion portion comprising a resilient deformable member (e.g., bulb, cone, cylinder, etc.) having a channel therethrough having a proximal opening sized to fit, during use of the device, snuggly against a manipulation rod or tube, and a relatively larger distal opening suitable to provide for angular and/or rotational movement of the rod or tube passing through the member via the distal and proximal openings while minimizing movement of the vaginal occlusion member when the tube is subjected to manipulation, and wherein the member is suitably configured to fit snugly against the contours of an inner vaginal tissue surface; and positioning, within a patient's vagina, the vaginal occlusion element between the cervix and the vaginal opening to provide for a snug fit within the vagina, wherein maintaining insufflation of the patient's abdomen during a surgical procedure is afforded, wherein such that the abdomen of the patient remains insufflated. In certain aspects, the method further comprises the use of a cervical fitting member in combination with the vaginal occlusion element, wherein the cervical fitting member is capable of being affixed to the cervix via barb means to preclude or retard slipping or rotating of the uterine occlusion element relative to the cervix. In certain aspects, the method further comprises deployment of the barb means of the cervical fitting member, wherein the cervical fitting member is prevented from slipping or rotating from the cervix. In certain embodiments, deployment of the barb means of the cervical fitting member allows for snug fitting which prevents slipping or rotating without stitching the cervical fitting portion into the cervix. In certain aspects, the surgery comprises laparoscopic surgery. In particular embodiments, the surgery comprises hysterectomy.

Other features and advantages of the invention will become apparent from the following detailed description, which illustrate, by way of example, various features of embodiments of the invention.

### BRIEF DESCRIPTION OF THE DRAWINGS

Exemplary embodiments are illustrated in referenced figures. It is intended that the embodiments and figures disclosed herein are considered illustrative rather than restrictive.

FIG. 1 is a prior art device as disclosed in United States Design Patent Number 421,497.

FIG. 2 is a prior art device as disclosed in U.S. Pat. No. 8,082,925.

FIG. 3 is a prior art device as disclosed in U.S. Pat. No. 5,520,698.

FIG. 4 is a side perspective view of one embodiment of the current inventive uterine manipulator.

FIGS. 5A-C are cross-sectional views of the embodiment of FIG. 4 in use.

FIG. 6 is a side perspective view of another embodiment of the current inventive uterine manipulator.

FIG. 7 is a cross-sectional view of the embodiment of FIG. 6 in use.

FIG. 8 is a side perspective view of another embodiment of the current inventive uterine manipulator, where the vaginal occlusion member is positioned away from the cervical fitting member.

FIGS. 9A-F are side perspective views of additional embodiments of the current inventive uterine manipulator, where the cervical fitting member is configured with barbs.

FIGS. 10A-B are side perspective views of additional embodiments of the current inventive uterine manipulator, where the vaginal occlusion member is shown as cylinders.

FIGS. 11A-C are side perspective views of additional embodiments of the current inventive uterine manipulator, where the vaginal occlusion member is shown as cones.

FIG. 12 is a side perspective view of additional embodiments of the current inventive uterine manipulator, where the vaginal occlusion member is shown as wagon wheel shape.

FIG. 13 is a side perspective view of additional embodiments of the current inventive uterine manipulator, where the vaginal occlusion member is shown as torus shape.

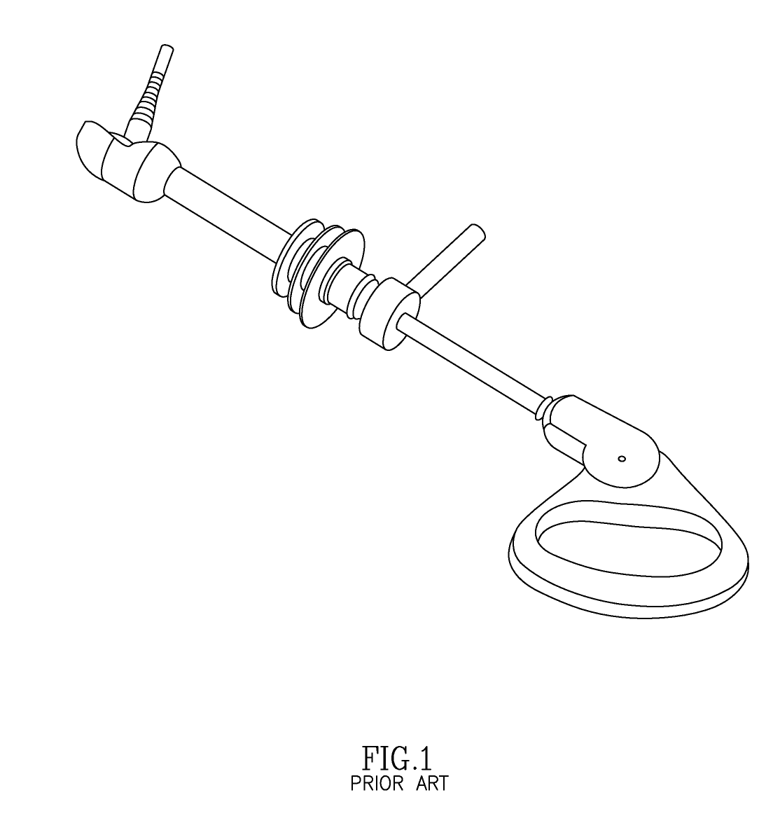

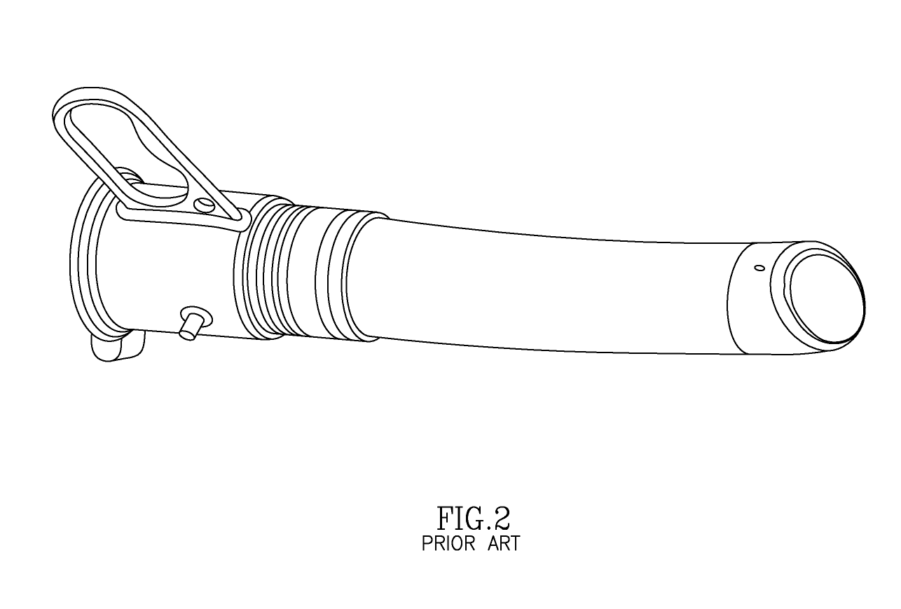

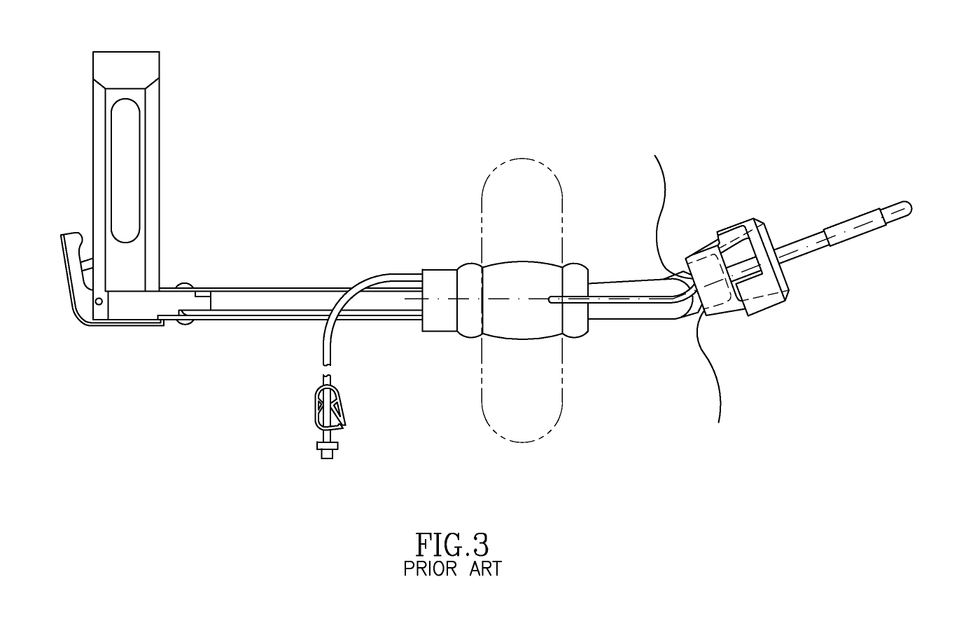

### DETAILED DESCRIPTION OF ASPECTS OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russell, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Occlusion” as used herein may include, but is in no way limited to any form of blocking, obstruction, or trapping of a liquid or gas within a cavity.

“Vaginal occluding element, portion, member or device” as used herein may include, but is in no way limited to, any element, portion, member or device that can occlude, block, obstruction, or trap liquid or gas within the abdominal area of a patient. Examples of shapes of vaginal occluding devices can include, but are not limited to spheres, ovoids, cylinders, cones, tori (toroidal), bulbs, rings, or wheels.

“Laparoscopic surgery”, also called minimally invasive surgery (MIS), bandaid surgery, keyhole surgery as used herein refers to a modern surgical technique in which operations in the abdomen are performed through small incisions (usually 0.5-1.5 cm), with use of tiny cameras.

“Insufflation” as used herein relates to the blowing of a powder, vapor, or gas into a body cavity. In particular, insufflation as used herein relates to the blowing of or otherwise introducing a gas into the abdomen to inflate the abdomen of a patient during a laparoscopic surgery.

“Pneumoperitoneum” as used herein relates to surgeons deliberately creating a pneumoperitoneum by insufflating the abdomen with a gas (e.g., carbon dioxide) in order to perform laparoscopic surgery.

“Barbs” as used herein relate to any element suitable to retain and hold the distal cervical fitting/securing portion, as described herein, to the cervix. Barbs can include, but are not limited to points, hooks, tips, spurs, spikes, spines, vacuum channels or some other type of element that is preferably sharp and narrow.

“Balloon” as used herein relates to any item that can be filled and expanded when fluid (e.g., gas or liquid) is placed within it.

“Tubing portion” as used herein relates to any element that can be used to link the balloon at the distal end of the uterine manipulator with the fluid inlet at the proximal exterior end. Examples of tubing portions can include, but are not limited to, a catheter, a line, a tube, a drip, a drain, a feed, and a pipe. Preferably, such portions are curved and somewhat flexible but sufficiently rigid and/or resilient to provide for translocation of movement from an exterior handle to the cervical fitting/securing portion and/or intra-usterine balloon.

“Resilient deformable member” as used herein relates to a member being made of any suitable material that is bendable, deformable, manipulatible, and soft to enable conforming to the contours of a contact surface, while yet able to spring back into shape after bending, stretching or being compressed to conform to such contact surface, to provide for maintaining with a certain amount of tension its original form. Examples of materials that can be used to make the resilient deformable member can include, but are not limited to, soft plastics, eslastomers, rubber polymers, silicones, and gel materials enclosed in pliable plastics.

According to certain embodiments, the design of the current inventive uterine occlusion member (cervical fitting/securing element) for use in a uterine manipulator allows for the displacement of the cervix away from the ureters, retracts the urinary bladder and defines the colpotomy incision. According the further embodiments, the manipulator tube preferably conforms to the angle of the sacral curve and allows for easy manipulation of the uterus. An inflatable balloon at the distal end of the tube is used to stabilize the manipulator tube within the uterine cavity and provide for manipulation of the uterus.

According to still further embodiments, the current inventive vaginal occlusion element comprises a unique vaginal sealing element that fits tight against the interior vaginal tissue to maintain an abdominal insufflation-preserving seal while also providing for angular movement/adjustment of the manipulator tube and uterus during a surgery (e.g., laparoscopic surgery for a hysterectomy).

Further embodiments comprise utilization of a balloon inserted into the uterus via a tube/rod through the cervix, which facilitates, along with the cervical fitting/securing element, movement/adjustment of the cervix and uterus by the tube/rod.

Further embodiments relate to advantages of the invention relative to conventional uteri manipulation devices provided by a unique device designed for complete removal of human uteri by laparoscopy, which further includes an element useful in securing the uterus and uterine cervix and manipulating them to allow an adequate exposure of all anatomic structures, and providing an optimal viewing angle of the vulnerable anatomical structures as the ureters and bladder, said new device also including an element for exposure of the fascia and sealing of the vagina when making the incision of the fascia, thereby allowing the surgical procedure to continue in a more secure manner and preventing the leakage of the pneumoperitoneum. A first preferred embodiment of the present invention comprises in combination a vaginal occlusion/sealing portion and a cervical fitting/securing portion. The vaginal occlusion portion comprising a resilient deformable vaginal sealing member having a channel therethrough and comprising a small proximal opening and a larger distal opening (or a smaller distal opening subtended by a deformable material; to provide for a distal deformable grommet opening), relative to the proximal opening, wherein, the larger distal opening (or deformable grommet opening) provides for a substantial degree (e.g., from at least 10 degrees, to at least 15 degrees, to at least 20 degrees, to at least 25 degrees, to at least 30 degrees, to at least 35 degrees, to at least 40 degrees, to at least 45 degrees, to at least 50 degrees or greater angle) of unhindered angular (relative the channel axis) movement of a tube passing through the member via the distal and proximal openings while minimizing movement of the member when the tube is subjected to manipulation (e.g., angular movement/adjustment), and wherein the vagomal sealing member is suitably configured to fit snugly against the contours of an inner vaginal tissue surface.

According to certain embodiments, the vaginal occlusion/sealing portion is configured to snuggly and intimately conform to the interior vaginal wall, thereby maintaining sealing and precluding escape of gas used to insufflate the abdomen. According to certain embodiments, the resilient deformable member of the vaginal occlusion/sealing portion can be constructed out of soft plastics, eslastomers, rubber polymers, silicones, and gel materials enclosed in pliable plastics or the like. According to further embodiments, the material used to construct the resilient deformable vaginal sealing member is soft enough not to tear or cut the interior vaginal tissue, but sturdy enough to hold its shape and intimately conform to the interior tissue of the vagina with the ultimate goal of maintaining insufflation of the abdomen despite movement and repositioning of the uterine manipulator during surgical procedures.

According the still further embodiments, the vaginal occlusion/sealing portion having a channel therethrough and comprising a small proximal opening and a larger distal opening (or a smaller distal opening subtended by a deformable material; to provide for a distal deformable grommet opening), allows for a tube or catheter to be inserted into the small proximal opening through the channel and out a larger distal opening (or out a smaller distal opening subtended by a deformable material; to provide for a distal deformable grommet opening). The large distal opening (or a smaller distal opening subtended by a deformable material; to provide for a distal deformable grommet opening) being configured to allow significant angular and rotational manipulation of the tubing, or semi-rigid rod surrounding the tubing, without moving the sealing member and thus precluding or minimizing leakage of the gas used to insufflate the abdomen.

According to even further embodiments, the cervical fitting/securing portion of the current invention fits snugly and/or tightly up against and/or within the cervix. In further preferred embodiments, the cervical fitting portion is barbed or otherwise suitably configured with cervical engagement/retaining elements, which allows the cervical fitting portion to slip into the cervix easily, but which will not allow it to slip out or rotate from the cervix. In still further preferred embodiments, a switch or spring exists on the exterior portion of the device and can be activated or manipulated to deploy the barbs (or other suitable means as described herein) in the distal cervical fitting portion to seat securely the distal cervical fitting portion into the cervix. In further preferred embodiments, the barbs (or other suitable means) on the cervical fitting portion are deployed to attach and/or adhere the cervical fitting portion to the cervix in such a way that any pushing, pulling and manipulation that occurs during the surgical procedure will not dislodge the device from the cervix and rather will remain secure and prevent leakage of the pneumoperitoneum in the absence of sowing or stitching the device into place on the cervix.

A second preferred embodiment of the present invention includes an insertion rod, which is preferably semi-rigid and optionally arched to fit the angle of the sacral curve, as part of a uterine occluder device as disclosed herein. Tubing runs throughout the length of the device or rod and connects a balloon at the distal end to a syringe at the proximal end. The syringe can inflate the balloon through the tubing using a liquid or gas (e.g., saline).

A third preferred embodiment of the present invention includes an insertion rod with integral handle extending perpendicularly or at a slight off-perpendicular angle at the proximal end of the rod. A manipulator tip for insertion into the uterus is mounted on the distal end of the insertion rod. The handle can manipulate the tip (e.g., rotate or inflation of the balloon). Tubing runs throughout the length of the device and connects a balloon at the distal end to the handle at the proximal end. The balloon can be inflated upon a particular manipulation of the handle. The balloon inflation can occur using a liquid or gas (e.g., saline).

A fourth preferred embodiment of the present invention comprises a vaginal occlusion/sealing portion, which comprises a resilient deformable sealing member having a channel therethrough and comprising a small proximal opening and a larger distal opening (or a smaller distal opening subtended by a deformable material; to provide for a distal deformable grommet opening), relative to the proximal opening, wherein, the larger distal opening (or the smaller distal opening subtended by a deformable material; to provide for a distal deformable grommet opening) provides for a significant amount (e.g., at least 10 to 50 degrees, or more) of substantially unhindered angular movement/adjustment of a tube passing through the member via the proximal and distal openings while minimizing movement of the sealing member relative to the contact vaginal tissue when the tube is subjected to manipulation, and wherein the member is suitably configured to conform and seal with the contours of an inner vaginal tissue surface.

According to certain embodiments, the unique vaginal occlusion/sealing portion is configured to be conformed to, and fit snuggly against the interior vaginal wall, thereby sealing a possible escape route for the gas used to insufflate the abdomen.

According to certain embodiments, the resilient deformable vaginal sealing member of the vaginal occlusion portion can be constructed out of soft plastics, eslastomers, rubber polymers, silicones, and gel materials enclosed in pliable plastics or the like. According to further embodiments, the material used to construct the resilient deformable vaginal sealing member is soft enough not to conform to and seal the vaginal opening but not tear or cut the interior vaginal tissue, and sufficiently resilient to hold its shape and fit snugly against the interior tissue of the vagina with the ultimate goal of maintaining insufflation of the abdomen.

According to still further embodiments, the vaginal occlusion/sealing portion having a channel therethrough and comprising a small proximal opening and a larger distal opening (or a smaller distal opening subtended by a deformable material; to provide for a distal deformable grommet opening), allows for a tube or catheter to be inserted into the small proximal opening through the channel and out a larger distal opening (or out the smaller distal opening subtended by a deformable material; to provide for a distal deformable grommet opening). The large distal opening (or the smaller distal opening subtended by a deformable material; to provide for a distal deformable grommet opening) is configured to allow significant angular and/or rotational manipulation/adjustment of the tubing, or semi-rigid rod surrounding the tubing, without allowing leakage of the gas used to insufflate the abdomen.

The present invention also comprises a manipulator tip which possesses a soft tip and balloon at the distal end thereof for firm retention of the tip within the uterine cavity. According to certain embodiments, there is a single lumen in combination with a syringe to inflate the balloon. Other embodiments related to a single lumen in combination with a pressure-actuated valve to inflate the balloon. The embodiments allow for a lumen within a small-profile, which can be inserted into the uterus of most patients with little or no cervical dilation.

The presently disclosed uterine manipulator for total removal of human uteri is an instrument designed based on experience gained by the use of different manipulators and knowledge of the advantages and shortcomings of each of them. From these shortcomings a uterine manipulator has been designed which meets certain basic requirements such as: an element for securing the uterus and cervix to allow an adequate exposure of all anatomic structures to provide an optimal angle of attack of vulnerable anatomical structures such as ureters and the bladder, and an element, or elements, for exposure of the fascia and sealing of the vagina, which allows for performing the procedure with adequate tissue exposure while preventing the escape of gas from the pneumoperitoneum. This is achieved in part by utilizing a unique vaginal sealing portion which is designed to fit snugly to the cervix and allow movement of the manipulation rod with minimal or no dislodgment of the vaginal seal.

In preferred embodiments, the cervical fitting/securing portion of the current invention fits snugly and/or tightly up against and/or within the cervix. In further preferred embodiments, the cervical fitting portion is barbed (or otherwise configured as described herein), which allows the cervical fitting portion to slip into the cervix easily, but which will not allow it to slip out or rotate from the cervix. In some aspects the barbs (or equivalent structures) can be engaged into the cervical tissue by twisting the device rod/exterior handle to lock in the barbs. In still further preferred embodiments, a switch or spring exists on the exterior portion of the device and can be activated or manipulated to deploy the barbs in the distal cervical fitting portion to seat securely the distal cervical fitting portion into the cervix. In further preferred embodiments, the barbs on the cervical fitting portion are deployed to attach and/or adhere the cervical fitting portion to the cervix in such a way that any pushing, pulling and manipulation that occurs during the surgical procedure will not dislodge the device from the cervix and rather will remain secure and prevent leakage of the pneumoperitoneum in the absence of sowing or stitching the device into place on the cervix.

It is preferred that each and/or all of the embodiments and elements of the vaginal occlusion device or uterine manipulator of the present invention be primarily fabricated so as to render them inexpensive and therefore disposable after a single use. With reference to the attached figures, the uterine manipulator 100 comprises an element to secure the cervix and uterus and to manipulate the cervix and uterus, and a tip portion useful to place in position the elements of the system that come into contact with the organ to be removed.

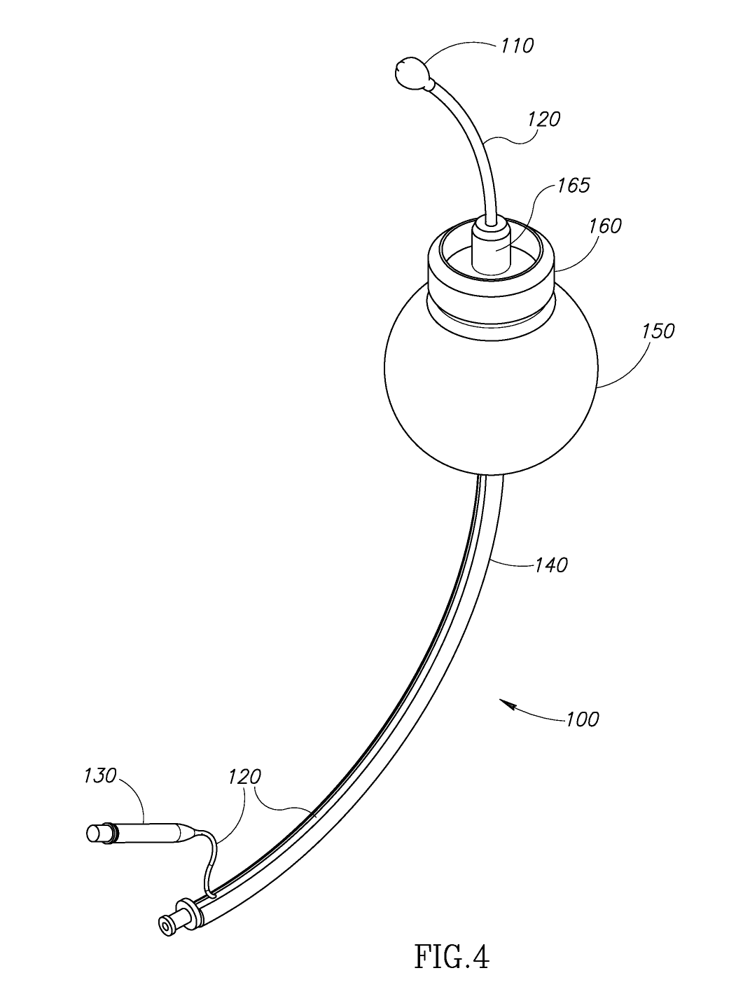

FIG. 4 shows a view of one embodiment of the inventive uterine manipulator 100. According to certain embodiments, at the distal end of the device there is a balloon 110 that is inserted into the uterus and can be inflated to allow for ease in manipulation of the uterus during surgery. Attached to the balloon is tubing 120 that extends throughout the entire length of the device 100. Attached to the tubing 120 at the proximal end of the tubing is a syringe 130 that can be filled with a gas or fluid to allow for inflating the balloon 110. The tubing 120 extends through a semi-rigid holder 140 that is flexible enough to adjust, but rigid enough to manipulate and hold its structure. The semi-rigid holder 140 (preferably slotted) communicates, abuts or attaches to a vaginal occluder or sealer 150 allowing the tubing to pass through from the semi-rigid holder 140 into and through the vaginal occluder 150. Optionally, the semi-rigid holder 140 contains a slot into which and out of which the tubing can be placed, slid along, and removed. According to certain embodiments, the slot for the tubing in the semi-rigid holder allows for adjusting the tubing and device as needed throughout surgery. The vaginal occluder 150, comprising a cervical proximal end and a vaginal proximal end, is rigid enough (e.g., resilient) to maintain its structure but flexible enough to be manipulatable and intimately conformable with the vaginal tissue. The cervix proximal end of the vaginal occluder 150 comprises or is configured to meet/fit with a cervix proximal element 160 that is configured to fit snug against the cervix and retain the cervix, while the vaginal proximal end of the vaginal occluder 150 is configured to fit snuggly against and conform to the contours of the inner vaginal tissue. According to certain embodiments, the vaginal occluder 150, comprising a cervical proximal end and a vaginal proximal end, can slide along the tubing 120, to allow for proper insertion into the vagina and tailored adjustment for different surgical subjects.

According to certain embodiments, a portion, or element of the cervical proximal element 160 that fits snug against the cervix can fit inside the cervix (e.g., a plug element), sit inside the cervix, and/or sit over the cervix (e.g., a cap with a plug). According to further embodiments, the cervical fitting proximal element 160 (e.g., the plug or cap) adheres and attaches to the cervix in a manner that any pushing, pulling or manipulation that occurs during the surgical procedure will not cause the element or distal cervical fitting portion (e.g., the plug or cap) to slip, move or become dislodged relative to the cervix. According to still further embodiments, this adherence and attachment occurs via any suitable means including but not limited to barbs, points, hooks, tips, spurs, spikes, spines, vacuum channels or some other type of element that is preferably sharp and narrowto affix the cervical proximal element 160 to the cervix. Moreover, this adherence can occur in the absence of sowing or stitching the element or distal cervical fitting portion (e.g., the plug or cap) into the cervix. In some aspects engagement or ‘setting’ of the cervix by said elements is by a twisting motion of the manipulator rod. According to still further embodiments this unique tight/snug fitting against both the vagina and cervix by the disclosed vaginal occluder 150 allows for the insufflation of the abdomen to be maintained during surgery.

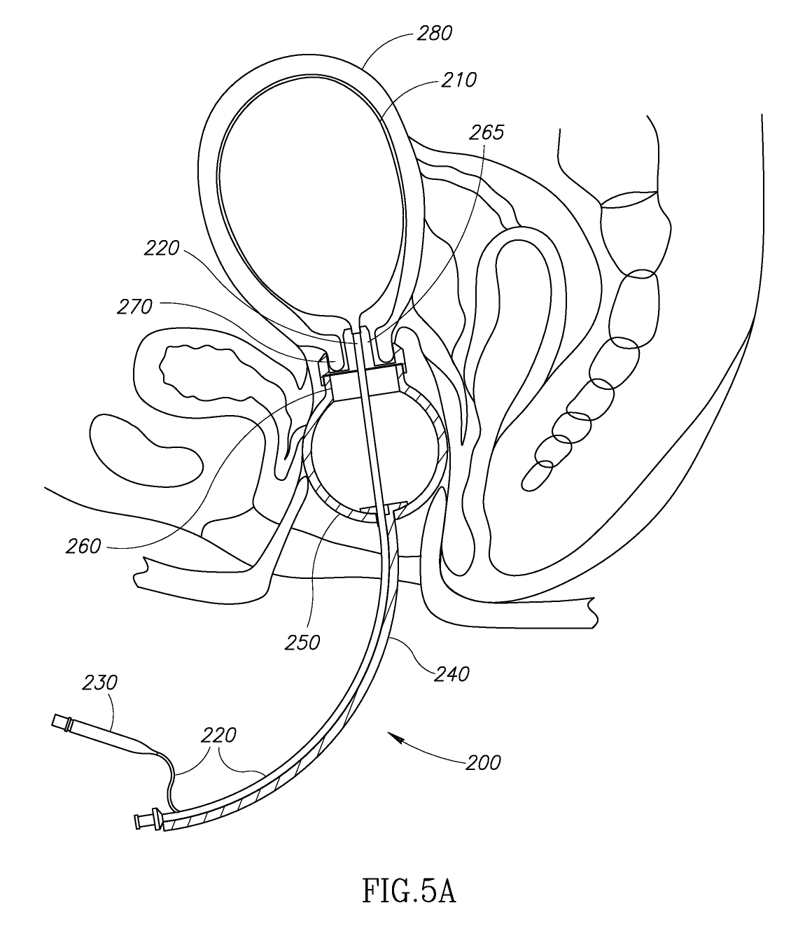

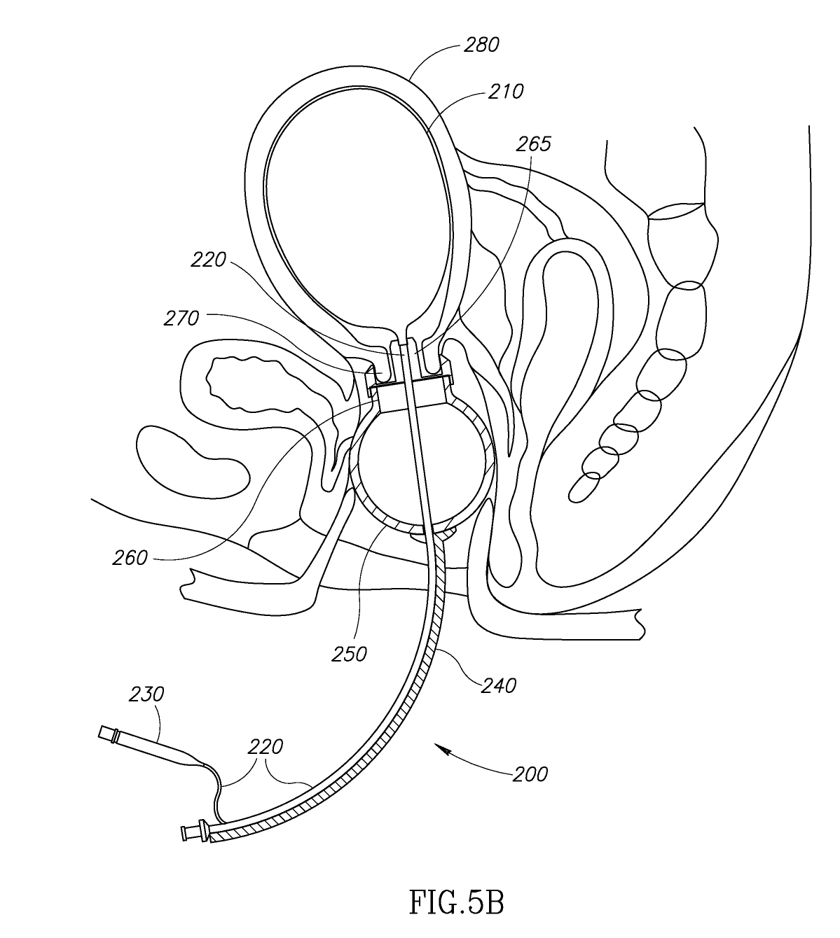

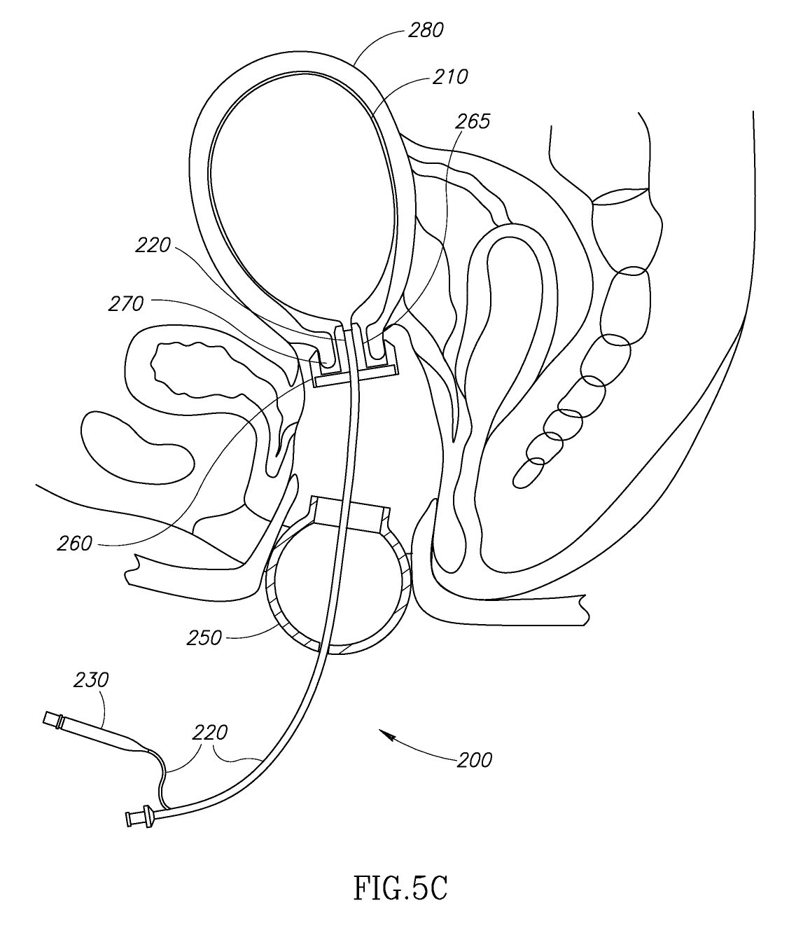

FIGS. 5A-C show respective cross-sections of the embodiments of the inventive uterine manipulator 200 of FIG. 4 in use. In particular, FIGS. 5A-C show the balloon 210 inflated in a uterus 280 of a surgical subject. This inflation allows for easier manipulation of the uterus during laparoscopic surgery. According to certain embodiments, the balloon 210 is inflated using a fluid or a gas that is fed to the balloon 210 via, for example, a syringe 230 attached to tubing 220. The tubing 220 in turn extends the entire length of the device 200. In addition, the figure shows that the cervical fitting portion 260 of the device fits snugly/tightly up against the cervix 270 to secure the cervix for manipulation. FIGS. 5A-B show optional attachment sites for the semi-rigid holder 240 to attach to the vaginal occlusion portion 250. FIG. 5C shows an additional embodiment of the uterine manipulator without the semi-rigid holder and also with the vaginal occluder 250 preferably positioned more proximal along the tube 220, where such positioning affords a tailored fit with the surgical subject. Note the larger distal channel opening of the vaginal sealing member (relative to the smaller snug-fitting proximal channel opening), which allows for angular movement/adjustment of the manipulation tube/rod with little or no concomitant movement and disruption of the vaginal occluder 250.

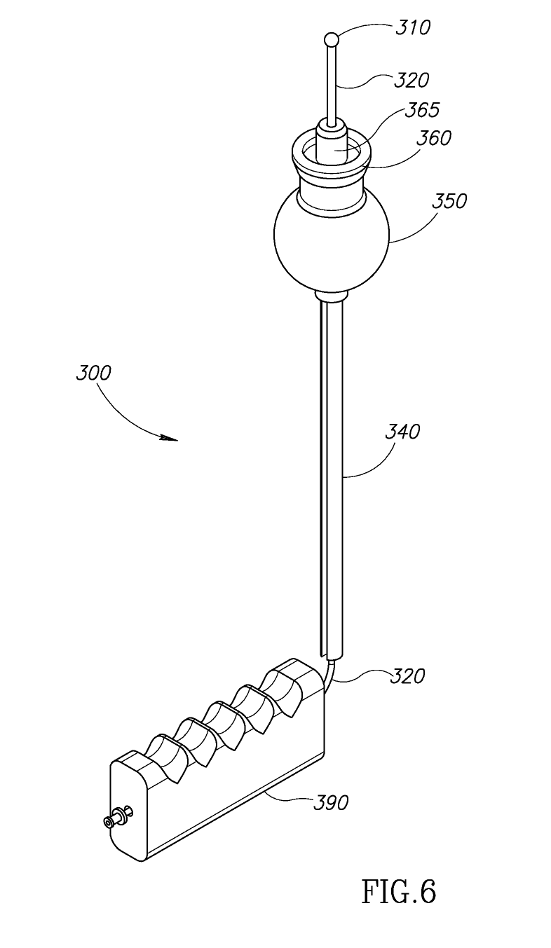

FIG. 6 shows another embodiment of the current inventive uterine manipulator 300. According to certain embodiments, at the distal end of the device 300 there is a balloon 310 that is inserted into the uterus and can be inflated to allow for ease in manipulation of the uterus during surgery. Attached to the tubing 320 at the proximal external end of the tubing 320 is a handle 390. According to certain embodiments, the handle 390 can be rotated or otherwise adjusted to allow for manipulation of the device 300 without breaking the seal formed between the vaginal proximal end of the vaginal occluder 350 (shown in this figure as adjustably abutting the cervical fitting/securing portion 360) and the contours of the inner vaginal tissue which keeps the abdomen insufflated. According to further embodiments, the handle 390 can be pulled or manipulated to inflate the balloon 310 attached to the tubing 320 at the distal end. Attached to the balloon 310 and handle 390 is tubing 320 that extends throughout the entire length of the device 300. The tubing 320 extends through a semi-rigid holder 340 that is flexible enough to adjust, but rigid enough to manipulate and hold its structure. The semi-rigid holder 340 (preferably slotted) communicates with, abuts or otherwise attaches to the vaginal occluder 350 allowing the tubing 320 to pass through from the semi-rigid holder 340 into the vaginal occluder 350. The vaginal occluder 350 is rigid enough (e.g., resilient) to maintain its structure but flexible enough to be manipulatable and intimately conformable to the vaginal tissue. The distal end (cervical proximate end) of the vaginal occluder 350 comprises (in particular embodiments) or is configured to adjustably meet with a cervix proximal element 360 that is configured to fit snug against the cervix, while the vaginal occluder 350 is configured to fit snug against and conform to the contours of the inner vaginal tissue. According to certain embodiments this unique tight/snug conforming fitting by the disclosed vaginal occluder 350 against both the cervix and inner vaginal tissue (either in the form of adjacent or separated cervical fitting and vaginal sealing elements) allows for the insufflation of the abdomen to be maintained without tearing the vaginal wall. According to further embodiments, the vaginal occluding element 350 can be constructed using a moldable, soft material that can be molded or conformed to the contours of the vaginal wall and that is soft enough to reduce vaginal wall tearing and injury during the surgical procedure when the uterine manipulator 300 is pushed, pulled and adjusted. The vaginal occlusion/sealing portion having a channel therethrough and comprising a small proximal opening and a larger distal opening (or a smaller distal opening subtended by a deformable material; to provide for a distal deformable grommet opening), allows for a tube or catheter to be inserted into the small proximal opening through the channel and out a larger distal opening (or out the smaller distal opening subtended by a deformable material; to provide for a distal deformable grommet opening). The large distal opening (or the smaller distal opening subtended by a deformable material; to provide for a distal deformable grommet opening) is configured to allow significant angular and/or rotational manipulation/adjustment of the tubing, or semi-rigid rod surrounding the tubing, without allowing leakage of the gas used to insufflate the abdomen.

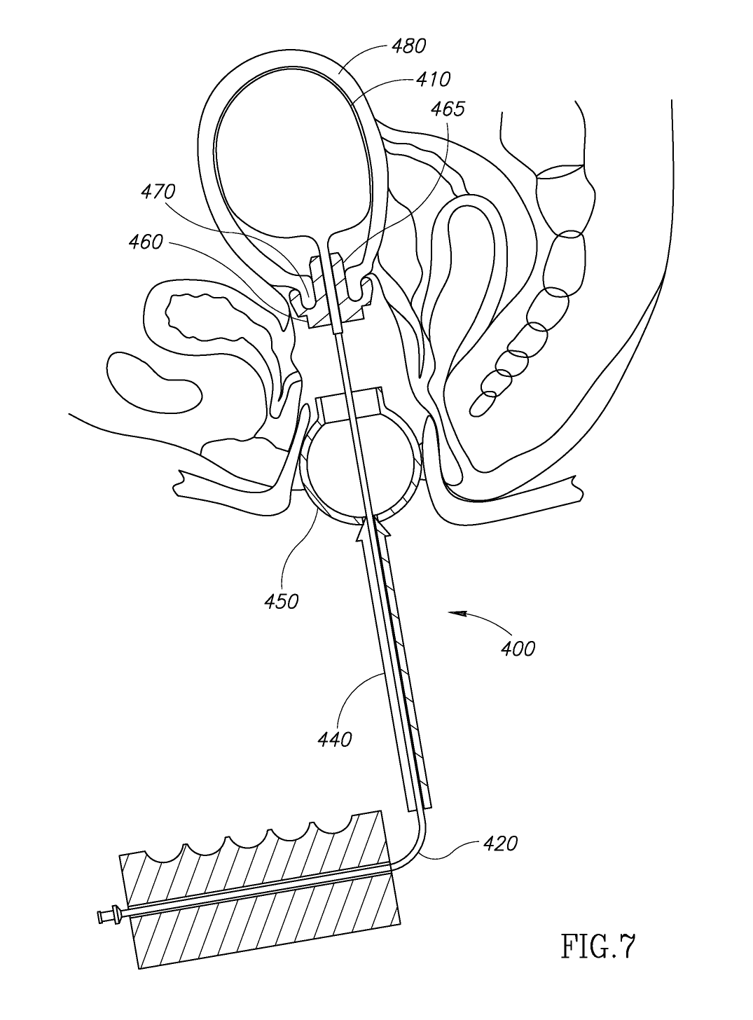

FIG. 7 shows a cross-section of the embodiment of the inventive uterine manipulator 400 of FIG. 6 in use. In particular, FIG. 7 shows the balloon 410 inflated in the uterus 480 of a surgical subject. This inflation allows for easier manipulation of the uterus 480 during laparoscopic surgery. According to certain embodiments, the balloon 410 is inflated using a fluid or a gas that is fed to the balloon via the tubing 420 and the adjustment (e.g., rotating or pulling) of the handle 490 attached to the tubing 420. The tubing 420 in turn extends the entire length of the device 400. In addition, the figure shows that the cervical fitting portions 460 and 465 fits snugly/tightly up against and/or within the cervix 470. According to certain embodiments, the vaginal occluder 450, while being positionable to meet or abut the cervical fitting portion, is configured to be adjustably positioned apart from the cervical fitting portion that fits snugly/tightly up against the cervix 470 and the vaginal tissue. According to further embodiments the cervical fitting portion is barbed (or otherwise configured), to allow for the device 400 to slip into the cervix 470 easily, but preventing dislodgement from or rotation relative to the cervix 470. According to further embodiments, the cervical fitting portion 460 may be rotationally “set” or anchored, or a switch or spring existing on the handle 490 can be activated or manipulated to deploy the barbs (or suitable elements) in the cervical fitting portion 460 to securely seat the cervical proximal cervical fitting portion into the cervix. According to still further embodiments, the barbs on the cervical proximal cervical fitting portion 460 are deployed to attach and/or adhere the cervical fitting portion to the cervix in such a way that any pushing, pulling and manipulation that occurs during the surgical procedure will not dislodge the device from the cervix and rather will remain secure in the absence of sowing or stitching the device into place on the cervix.

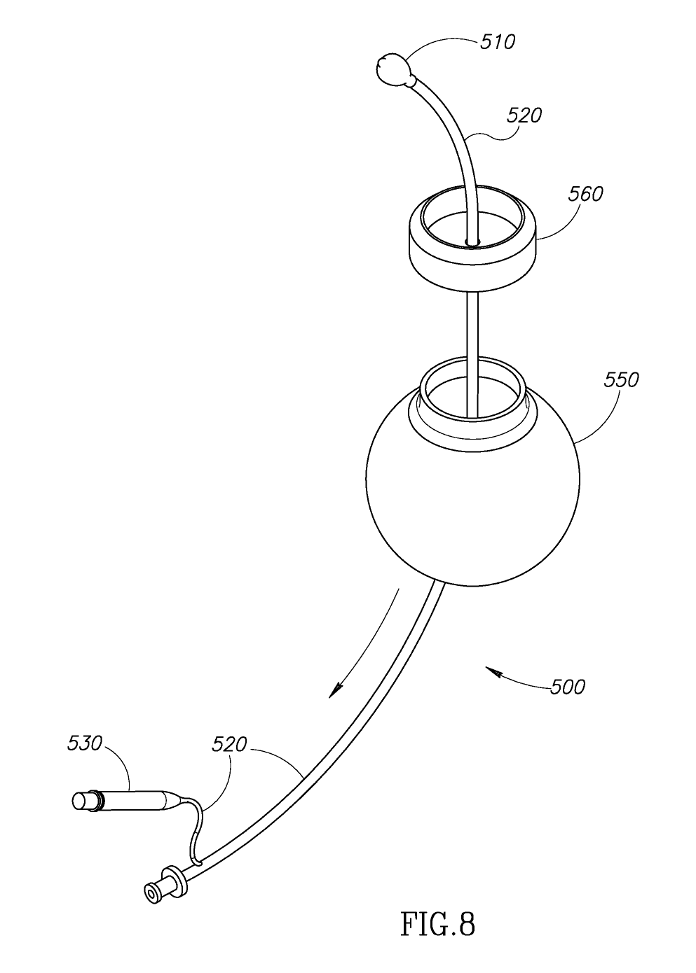

FIG. 8 shows that the vaginal occluder 550 is movable and positionable along the tubing 520. In particular, FIG. 8 shows that the vaginal occluder 550 is adjustably positionable in the vaginal proximal direction, away from the cervical fitting portion 560. The cervical fitting portion 560 is shown in this embodiment without the optional plug element that this insertible into the cervix.

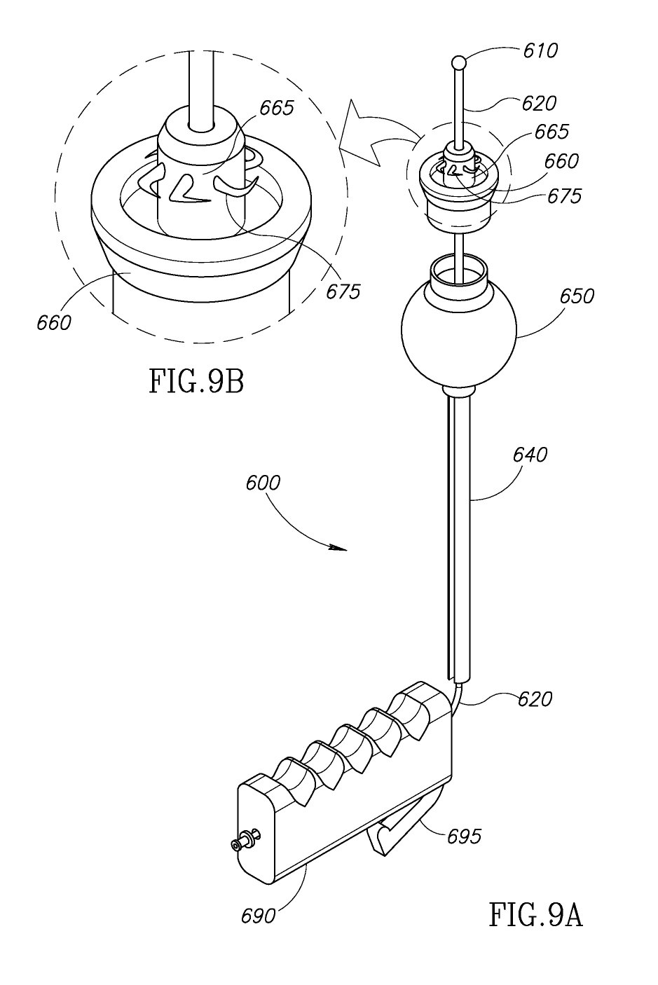

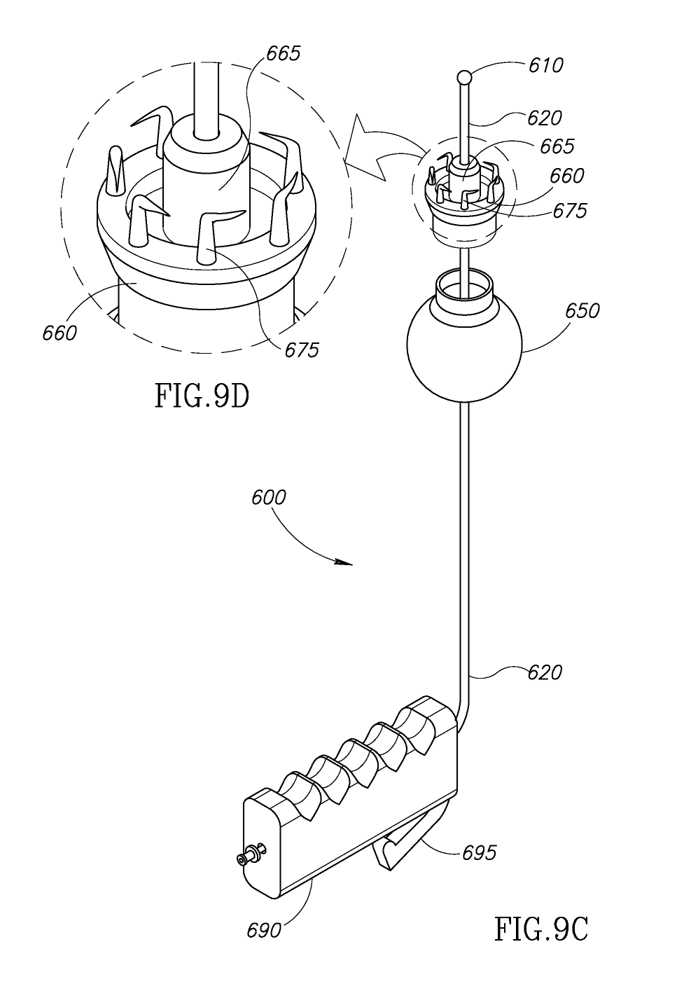

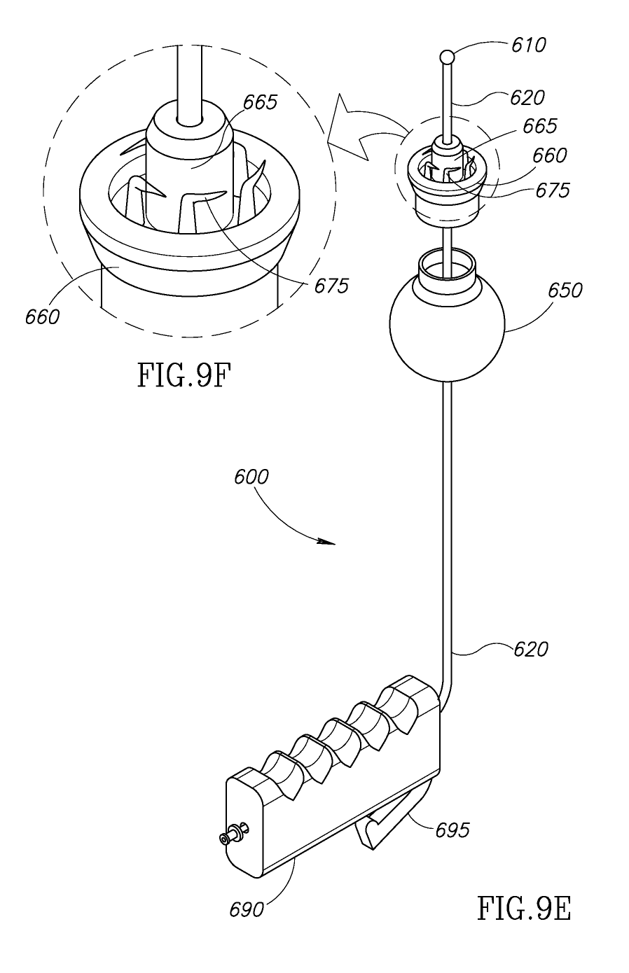

FIGS. 9A-F show variations in positioning of the optional barbs 675 on the plug portion of the cervical fitting portion 665 of the uterine manipulator. Further, FIG. 9A and 9B show that the barbs 675 optionally can be positioned on the cervical fitting portion that is positioned within the cervix 665. Alternative embodiments show that the barbs 675 optionally can be positioned on the cervical fitting portion that abuts to the cervix 660 (FIGS. 9C-F). Alternatively, combinations of such positioning can be used. In some embodiments the directions of the barbs are aligned as shown. In alternate embodiments the directions/angles of at least some of the barbs may be opposed or different. FIGS. 9A, 9C and 9E also use of a more conventional trigger-type handle as used in the art.

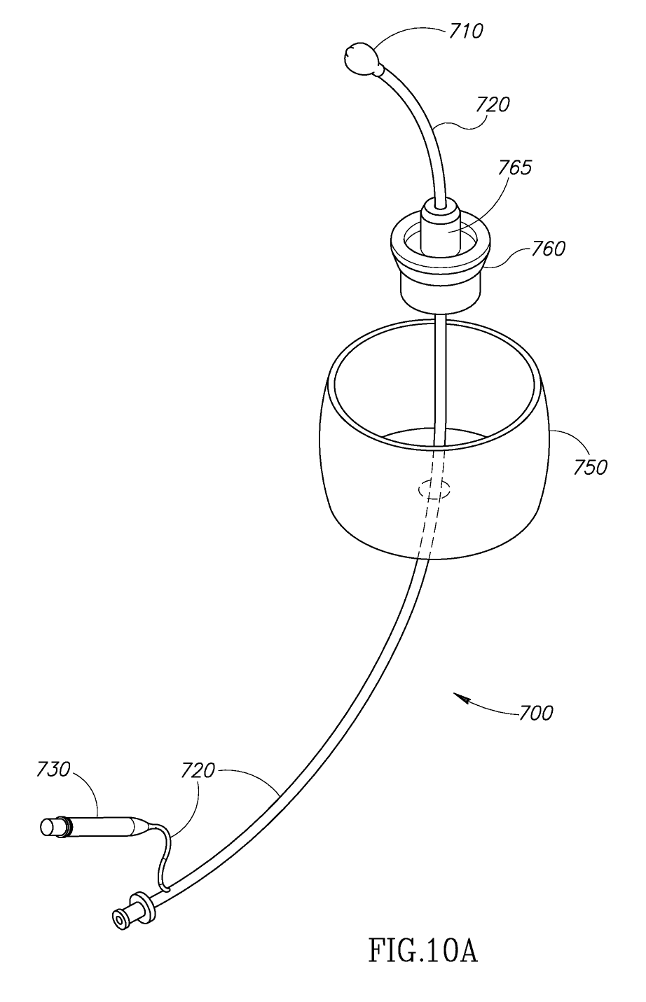

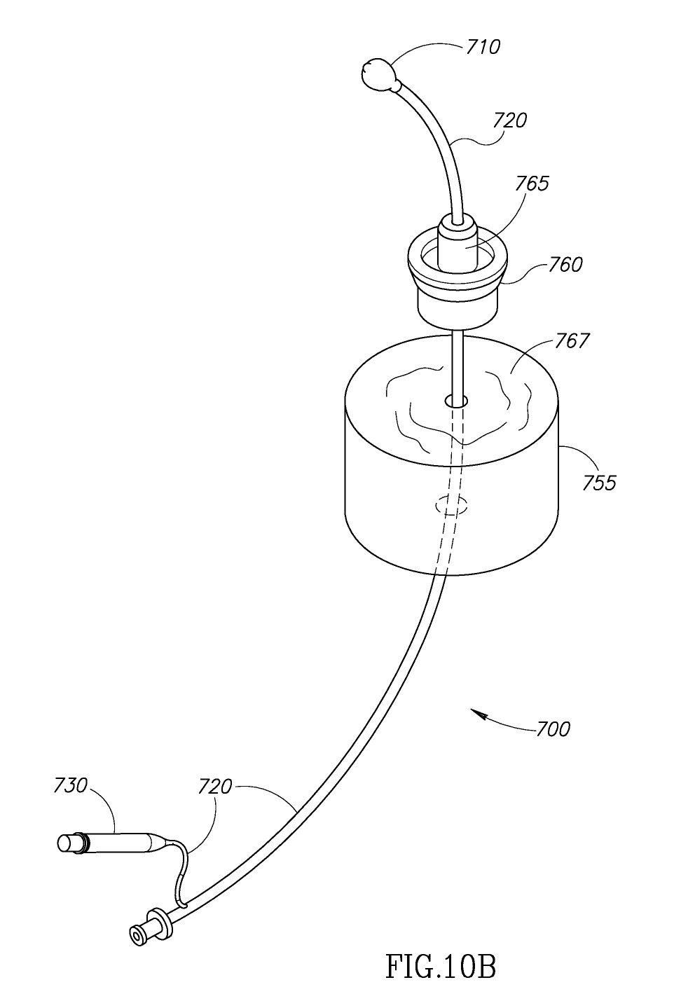

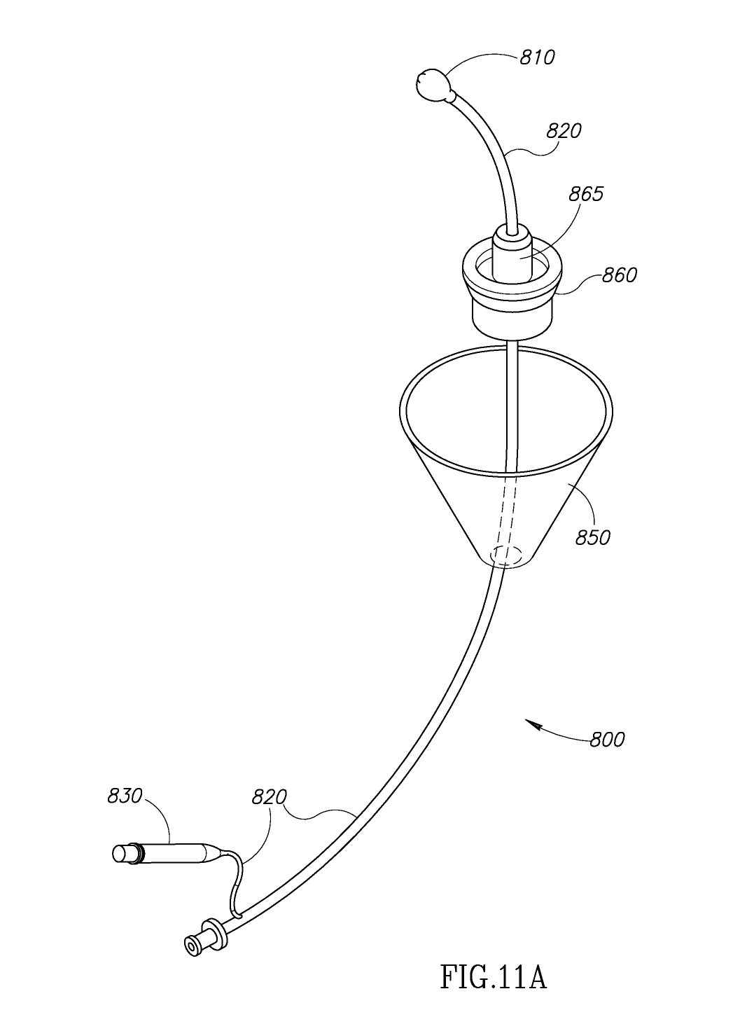

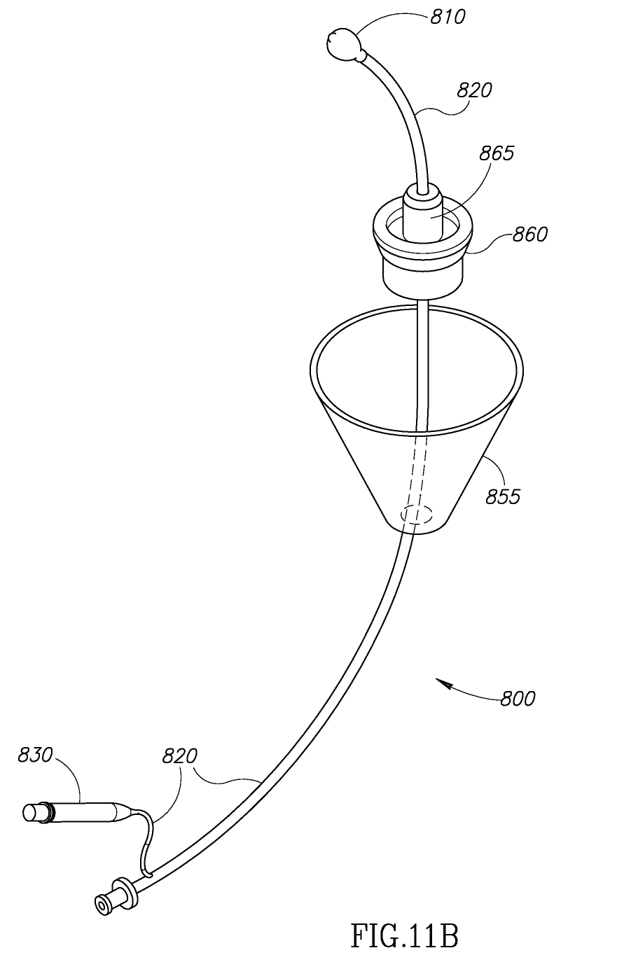

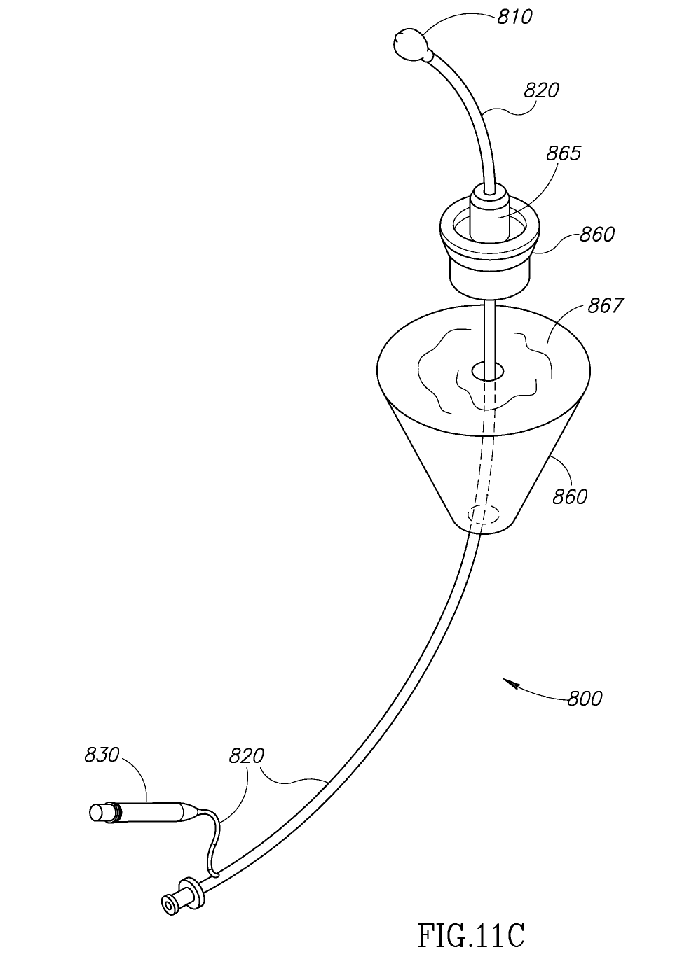

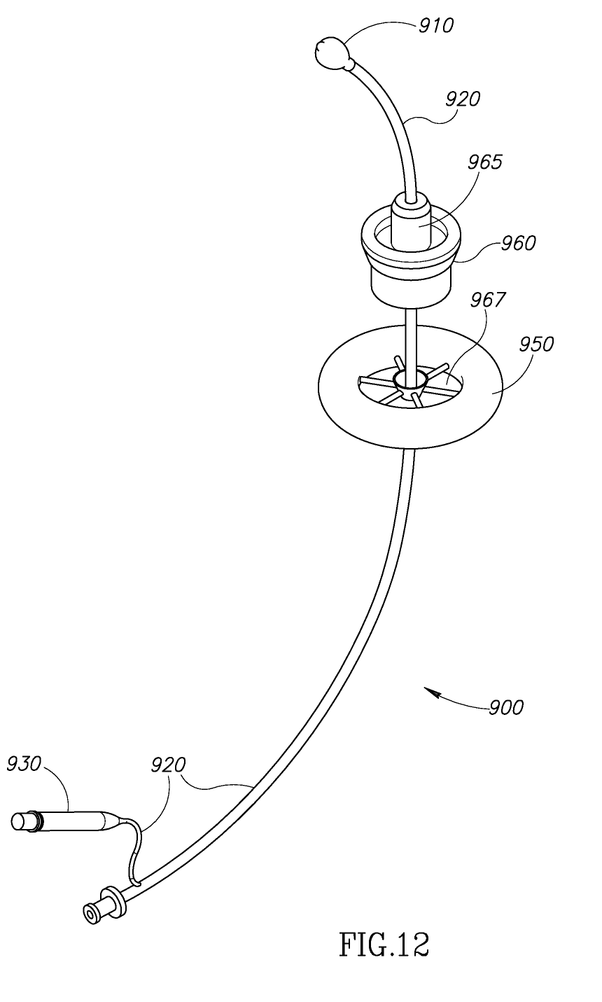

FIGS. 10A-B, 11A-C, and 12 show alternative embodiments for the shape of the resilient vaginal occluder/sealer element. Preferably the vaginal occluder/sealer element is generally bulbous or spherical in shape (FIGS. 4-9). Alternately, the vaginal occluder/sealer element has the shape of a cylinder (FIGS. 10A-B), cone (FIGS. 11A-C) or torus with spokes (FIG. 12). Preferably, the resilient vaginal occluder/sealer element comprises an axial channel having a small opening at the proximal end for the tube to slip into and a larger opening (or a smaller distal opening subtended by a deformable material; to provide for a distal deformable grommet opening), at the distal end that allows for a tube or catheter to be inserted into the small proximal opening through the channel and out a larger distal opening (or out the smaller distal opening subtended by a deformable material; to provide for a distal deformable grommet opening). The large distal opening (or the smaller distal opening subtended by a deformable material; to provide for a distal deformable grommet opening) is configured to allow significant angular and/or rotational manipulation/adjustment of the tubing, or semi-rigid rod surrounding the tubing, without allowing leakage of the gas used to insufflate the abdomen. One of skill in the art will, based on the present teachings, recognize that other shapes having these general characteristics and formed of suitable material may be used in practicing the invention.

Particular exemplary resilient shapes also show that they can have substantially or completely open distal ends (FIGS. 10A, 11A, and 11B) or may optionally be closed with a small hole (FIGS. 10B and 11C). In the closed distal end option, the material used to make the closed end is preferably made from a soft flexible rubber or plastic material that has low resistance to movement (e.g., is stretchable) (e.g., a smaller distal opening subtended by a deformable material; to provide for a distal deformable grommet opening) and as such allows the tube or semi-rigid rod to be angularly and/or rotational manipulated during a surgical procedure without disrupting the vaginal seal leading to leakage of gas from the abdomen.

Further, FIG. 12 shows an exemplary resilient torus with exemplary spokes that can optionally be used in place of the bulb. Using this shape the proximal end of the toroidal elements is closed to prevent passage of gas to keep the abdomen insufflated.

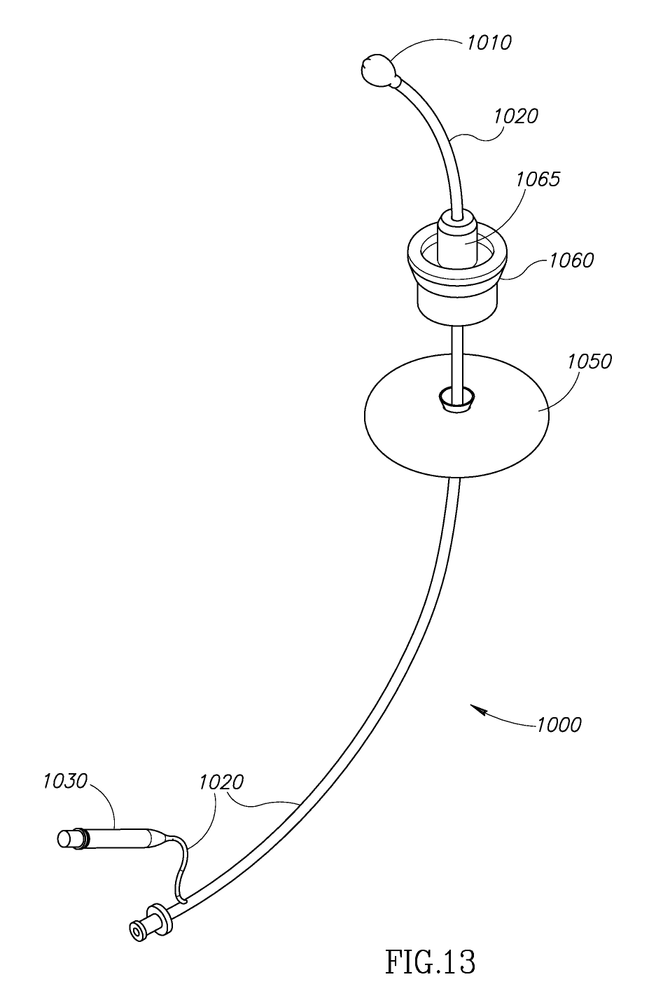

FIG. 13 shows a torus shaped embodiment of the inventive vaginal occlusion member.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

1. A vaginal occlusion device, comprising in combination:
a vaginal occlusion portion comprising a resilient deformable member (e.g., bulb, cone, cylinder, etc.) having a channel therethrough having a proximal opening sized to fit, during use of the device, snuggly against a manipulation rod or tube, and a relatively larger distal opening suitable to provide for angular and/or rotational movement of the rod or tube passing through the member via the distal and proximal openings while minimizing movement of the vaginal occlusion member when the tube is subjected to manipulation, and wherein the member is suitably configured to fit snugly against the contours of an inner vaginal tissue surface, and
a cervical fitting member configured to, in use of the device, engage and secure a cervix.

2. The device of claim 1, wherein, in use of the device, the vaginal occlusion portion is axially slidably positionable away from the cervical fitting member along the rod or tube.

3. The device of claim 2, wherein at least a part of the tubing portion is rigid.

4. The device of claim 1, wherein the cervical fitting member is configured to fit snugly against the cervix and secure the cervix.

5. The device of claim 4, wherein securing the cervical fitting member occurs via barb means, wherein the barb means are configured to preclude or retard slipping or rotating of the cervical fitting member relative to the cervix.

6. The device of claim 1, wherein the resilient deformable member generally comprises at least one shape selected from the group consisting of a sphere, a cylinder, a cone, a torus, a bulb, and a wheel.

7. A uterine manipulator suitable for laparoscopic surgical procedures, comprising:
a vaginal occlusion portion comprising a resilient deformable member (e.g., bulb, cone, cylinder, etc.) having a channel therethrough having a proximal opening sized to fit, during use of the device, snuggly against a manipulation rod or tube, and a relatively larger distal opening suitable to provide for angular and/or rotational movement of the rod or tube passing through the member via the distal and proximal openings while minimizing movement of the vaginal occlusion member when the tube is subjected to manipulation, and wherein the member is suitably configured to fit snugly against the contours of an inner vaginal tissue surface;
a cervical fitting member configured to be securable, in use, to a cervix; and
a balloon at a distal trans-cervical end of a tubing portion that extends through the vaginal occlusion element and the cervical fitting element, the tubing in fluid communication between a fluid inlet at a proximal exterior end and the balloon to enable intrauterine inflation of the balloon with a fluid, wherein the vaginal occlusion element is positioned or positionable along the tubing portion between the cervix and the vagina to provide for a snug fit with the vagina, thereby preventing leakage of pneumoperitoneum during a surgical procedure.

8. The device of claim 7, wherein securing the cervical fitting member occurs via barb means, wherein the barb means are configured to preclude or retard slipping or rotating of the vaginal occlusion device relative to the cervix.

9. The device of claim 8, wherein the deployment of the barbs allows for a secure fitting tight up against and/or within the cervix.

10. The device of claim 9, wherein a proximal exterior end of the device comprises a handle.

11. The device of claim 9, wherein the handle is in mechanical communication with the cervical fitting member.

12. The device of claim 11, wherein the handle is configured to deploy or otherwise manipulate or position the barb means of the cervical fitting member, wherein the barb means prevent or retard slipping or rotating of the device from placement, and wherein, the handle is in mechanical communication with the cervical fitting member.

13. The device of claim 12, wherein the deployment of the barb means is controlled by a switch or a spring.

14. The device of claim 9, wherein the resilient deformable member comprises the shape of at least one shape selected from the group consisting of a cylinder, a cone, a torus, a bulb, and a wheel.

15. The device of claim 9, wherein the surgery comprises laparoscopic surgery.

16. The device of claim 15, wherein the surgery comprises hysterectomy.

17. A vaginal occlusion device, comprising:
a vaginal occlusion portion comprising a resilient deformable member (e.g., bulb, cone, cylinder, etc.) having a channel therethrough having a proximal opening sized to fit, during use of the device, snuggly against a manipulation rod or tube, and a relatively larger distal opening suitable to provide for angular and/or rotational movement of the rod or tube passing through the member via the distal and proximal openings while minimizing movement of the vaginal occlusion member when the tube is subjected to manipulation, and wherein the member is suitably configured to fit snugly against the contours of an inner vaginal tissue surface.

18. The device of claim 17, in combination with a cervical fitting member suitable to secure a cervix and a uterine manipulation rod or tube.

19. The device of claim 18, wherein the vaginal occlusion portion is positionable along the rod or tube.

20. The device of claim 18, wherein at least a part of the rod or tubing portion is rigid.

21. The device of claim 18, wherein the cervical fitting member is configured to fit snugly against the cervix and secure the cervix using barb means.

22. The device of claim 21, wherein securing the cervical fitting member occurs via barbs, wherein the barbs are configured to preclude or retard slipping or rotating of the vaginal occlusion device relative to the cervix.

23. The device of claim 17, wherein the resilient deformable member comprises a shape selected from at least one from the group consisting of a cylinder, a cone, a torus, a bulb, and a wheel.

24. A method of conducting surgery, comprising:
obtaining a vaginal occlusion portion comprising a resilient deformable member (e.g., bulb, cone, cylinder, etc.) having a channel therethrough having a proximal opening sized to fit, during use of the device, snuggly against a manipulation rod or tube, and a relatively larger distal opening suitable to provide for angular and/or rotational movement of the rod or tube passing through the member via the distal and proximal openings while minimizing movement of the vaginal occlusion member when the tube is subjected to manipulation, and wherein the member is suitably configured to fit snugly against the contours of an inner vaginal tissue surface; and
positioning, within a patient's vagina, the vaginal occlusion element between the cervix and the vaginal opening to provide for a snug fit within the vagina, wherein maintaining insufflation of the patient's abdomen during a surgical procedure is afforded, wherein such that the abdomen of the patient remains insufflated.

25. The method of claim 24, further comprising the use of a cervical fitting member in combination with the vaginal occlusion element, wherein the cervical fitting member is capable of being affixed to the cervix via barb means to preclude or retard slipping or rotating of the uterine occlusion element relative to the cervix.

26. The method of claim 25, further comprising deployment of the barb means of the cervical fitting member, wherein the cervical fitting member is prevented from slipping or rotating from the cervix.

27. The method of claim 26, wherein the deployment of the barb means of the cervical fitting member allows for snug fitting which prevents slipping or rotating without stitching the cervical fitting portion into the cervix.

28. The method of claim 27, wherein the surgery comprises laparoscopic surgery.

29. The method of claim 28, wherein the surgery comprises hysterectomy.
